(19) United States
(12) Patent Application Publication
(10) Pub. No.: US 2005/0265890 A1
(43) Pub. Date: Dec. 1, 2005

(54) Washing machine having deodorizing filter and deodorizing method thereof

(75) Inventors: Hye Soon Yang, Yongin-Si (KR); Byoung Yull Yang, Ansan-Si (KR); Seong Min Oak, Masan-City (KR); Jae Ryong Park, Suwon-Si (KR); Sang Yeon Pyo, Suwon-Si (KR); Hyung Gyoon Kim, Suwon-Si (KR)

Correspondence Address: STAAS & HALSEY LLP, SUITE 700, 1201 NEW YORK AVENUE, N.W., WASHINGTON, DC, 20005, US

(73) Assignee: SAMSUNG ELECTRONICS CO., LTD., Suwon-si (KR)

(21) Appl. No.: 11/075,240

(22) Filed: Mar. 9, 2005

(30) Foreign Application Priority Data
May 31, 2004 (KR) 2004-39315

(51) Int. Cl. A61L 9/16; B08B 3/12

(52) U.S. Cl. 422/5; 422/121; 422/122; 68/3 R; 68/5 R

(57) ABSTRACT

A washing machine, which removes offensive odor from laundry without operating wash, rinse and/or dehydration modes, and a deodorizing method thereof. The washing machine includes a deodorizing tub, into which objects to be deodorized are put, a duct communicating with the deodorizing tub and serving as a passage for air containing odor particles separated from the objects in the deodorizing tub, and a deodorizing unit for removing the odor particles from the air passing through the duct. The deodorizing method includes separating odor particles from objects to be deodorized, and removing the separated odor particles by filtering out the separated odor particles with a deodorizing filter.

### CROSS-REFERENCE TO RELATED APPLICATION

This application claims the benefit of Korean Patent Application No. 2004-39315, filed May 31, 2004, in the Korean Intellectual Property Office, the disclosure of which is incorporated herein by reference.

### BACKGROUND OF THE INVENTION

1. Field of the Invention

The present invention relates to a washing machine having a deodorizing filter and method of deodorizing clothes in the washing machine, and, more particularly, to a deodorizing structure of a washing machine, by which particles that generate offensive odors, such as the smell of food or tobacco, and that permeate clothes are removed without an additional washing operation.

2. Description of the Related Art

A drum-type washing machine obtains washing power by force generated by the free falling of laundry in a rotating drum, and has a long washing time but reduces damage to the laundry and water consumption compared to a conventional pulsator-type washing machine. As a result, drum-type washing machines are increasingly being used now.

Such a drum-type washing machine has various modes such as wash, rinse, dehydration and dry, and is constructed such that the above modes are selectively combined. In cases in which the conventional washing machine is used to remove offensive odors, such as the smell of food or tobacco, from laundry, the laundry must be washed, thereby causing waste of electricity and water and requiring a long time to wash the laundry.

### SUMMARY OF THE INVENTION

Therefore, an aspect of the invention provides a washing machine, which removes offensive odor from laundry without a need to operate wash, rinse and/or dehydration modes, and a deodorizing method thereof.

In accordance with one aspect, the present invention provides a washing machine comprising: a deodorizing tub, into which objects to be deodorized are put; a duct that communicates with the deodorizing tub and serves as a passage through which air containing odor particles that have been separated from the objects in the deodorizing tub; and a deodorizing unit to remove the odor particles from the air passing through the duct.

The deodorizing unit may include a deodorizing filter to filter out the odor particles.

The deodorizing unit may further include a photocatalyst to perform a photocatalystic action to oxidize the odor particles in the deodorizing filter. The photocatalyst may comprise UV-LED lamps.

The duct, which is in communication with the deodorizing tub, may circulate the air inside the deodorizing tub, and a branch duct may be branched from the duct such that a part of the circulated air passes through the deodorizing unit and communicates with the deodorizing unit.

The branch duct may be provided with a damper to open and close the branch duct.

The deodorizing unit may further include a filter case to form a passage through which the air may flow along the deodorizing filter so that the air containing the odor particles that is introduced from the branch duct is purified by the deodorizing filter; and the filter case may be provided with an outlet, through which the air that is purified by the deodorizing filter is exhausted to the outside of the washing machine.

The deodorizing unit may be installed on a door of the washing machine.

The duct may include a condensing duct to communicate with a rear surface of the deodorizing tub, and a blowing duct, to communicate with the condensing duct, to be positioned on an upper surface of the deodorizing tub. The blowing duct may be provided with an air blast fan to generate power so as to circulate the air passing through the blowing duct. The duct may also include a heater to heat the air passing through the blowing duct. The branch duct may be branched from the blowing duct.

The deodorizing filter may be a ceramic filter and/or an activated carbon filter.

The deodorizing filter may be coated with the photocatalyst.

In accordance with another aspect of the invention, the present invention provides a method of deodorizing clothes in a washing machine comprising separating odor particles from objects to be deodorized; and removing the separated odor particles by filtering out the separated odor particles with a deodorizing filter.

The separating may be achieved by one or more methods of supplying hot draft to the objects to be deodorized, applying mechanical impact to the objects to be deodorized, or dissolving the odor particles in moisture in mist form supplied to the objects to be deodorized.

In the removing, the air in the deodorizing tub, in which the separated odor particles float, may be circulated into a duct that communicates with the deodorizing tub, and a part of the circulated air containing the separated odor particles are purified by the deodorizing filter.

The deodorizing filter may include a photocatalyst top perform a photocatalystic action to oxidize the odor particles in the deodorizing filter.

The air purified by the deodorizing filter may be exhausted to the outside of the washing machine.

Additional and/or other aspects and advantages of the invention will be set forth in part in the description which follows and, in part, will be obvious from the description, or may be learned by practice of the invention.

### BRIEF DESCRIPTION OF THE DRAWINGS

These and/or other aspects and advantages of the invention will become apparent and more readily appreciated from the following description of the embodiments, taken in conjunction with the accompanying drawings of which:

FIG. 1 is a perspective view of a washing machine having a deodorizing filter in accordance with a preferred embodiment of the present invention;

FIG. 2 is a longitudinal-sectional view of the washing machine of FIG. 1;

FIG. 3 is an exploded perspective view of the deodorizing filter of the washing machine of FIG. 1; and

FIG. 4 is a schematic view illustrating a deodorizing operation of the washing machine of FIG. 1.

### DETAILED DESCRIPTION OF THE PREFERRED EMBODIMENTS

Reference will now be made in detail to the embodiments of the present invention, examples of which are illustrated in the accompanying drawings, wherein like reference numerals refer to the like elements throughout. The embodiments are described below to explain the present invention by referring to the figures.

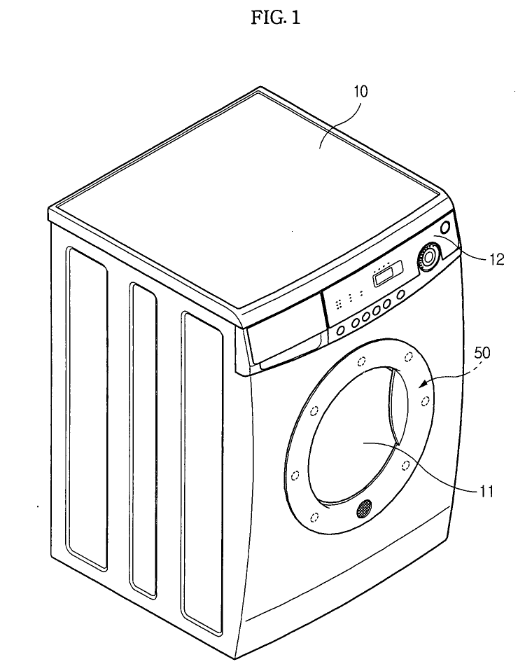

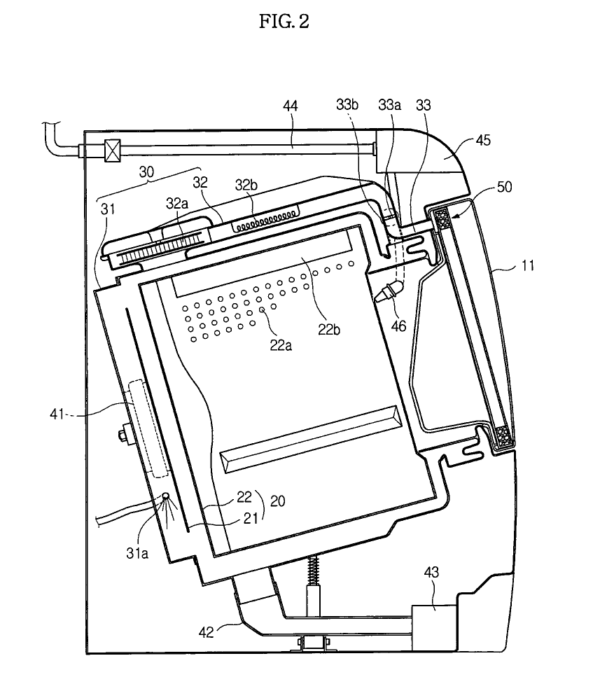

As shown in FIGS. 1 and 2, a washing machine of the present invention comprises a housing 10 to define an external appearance of the washing machine, a door 11, installed on a front surface of the housing 10, which opens and closes the front surface of the housing 10 so that laundry to be deodorized may be placed into the washing machine therethrough, and an operating unit 12 installed on the front surface of the housing 10 above the door 11 to manipulate the operation of the washing machine.

A cylindrical drum 20 provided with a top surface that is opened toward the door 11 is placed in the housing 10. The drum 20 serves as a deodorizing tub during a deodorizing process, and has a double structure including a fixed tub 21 and a rotary tub 22. Here, the rotary tub 22 is rotatably installed in the fixed tub 21, and is operated by a driving motor 41 installed on a rear surface of the fixed tub 21. The rotary tub 22 includes a plurality of through holes 22a formed through side and rear surfaces thereof so that the rotary tub 22 communicates with the fixed tub 21 through the through holes 22a, and a plurality of lifters 22b installed on the inner circumference thereof in a longitudinal direction to lift the laundry.

A water feed pipe 44 to supply water to the inside of the drum 20 is installed above the upper surface of the fixed tub 21. One end of the water feed pipe 44 is connected to the rear surface of the housing 10 to receive water supplied from an external water supply source (not shown), and the other end of the water feed pipe 44 is connected to a detergent container 45 installed in the front surface of the housing 10. A water feed nozzle 46, to spray water to the inside of the rotary tub 22 to supply water having passed through the detergent container 45 to the inside of the drum 20, is connected to the detergent container 45. A drain pipe 42 and a drain pump 43, to discharge the used water to the outside, are installed on the lower part of the fixed tub 21.

A duct 30 to take air from the inside of the drum 20 and then re-circulate the air into the drum 20 is formed on the outer circumference of the fixed tub 21. The duct 30 is divided into a condensing duct 31, which is installed on the rear surface of the fixed tub 21 in a longitudinal direction and communicates with a lower part of the fixed tub 21, and a blowing duct 32, which communicates with the upper end of the deodorizing duct 31, extends along the upper surface of the fixed tub 21, and then communicates with the inside of the drum 20 at the upper part of the front surface of the fixed tub 21.

The condensing duct 31 condenses moisture in air having a high temperature and a high humidity that is introduced from the drum 20 in the dehydration mode. and the condensing duct 31 is provided with a condensate water nozzle 31 a to spray cold water to the air passing through the condensing duct. The blowing duct 32 is provided with an air blast fan 32a to generate power so as to circulate air between the drum 20 and the duct 30, and a heater 32b to heat the air that is introduced into the drum 20.

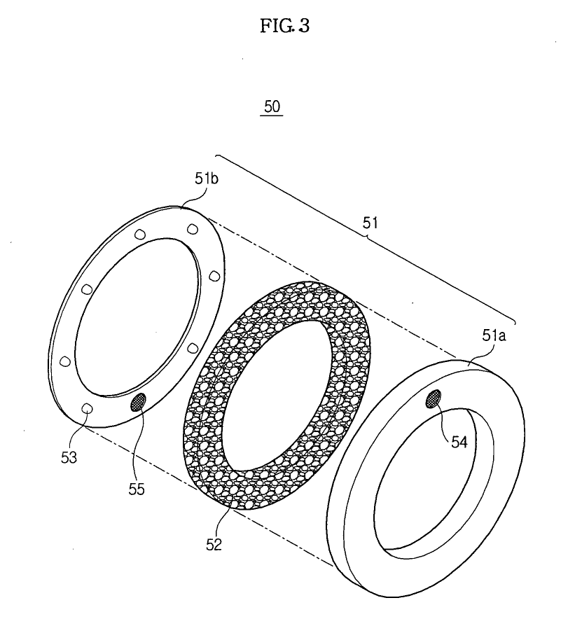

A deodorizing unit 50 having a circular loop shape is installed along the circumference of a transparent window formed in the door 11 so as to allow a viewing of the inside of the drum 20. The deodorizing unit 50 ssucks odor particles that are separated from objects to be deodorized. A detailed structure of the deodorizing unit 50 is shown in FIG. 3.

The deodorizing unit 50 includes a filter case 51 in a circular loop shape having a hollow formed therethrough, and a deodorizing filter 52 installed in the filter case 51.

A conventionally well-known filter, such as a ceramic filter or an activated carbon filter, is used as the deodorizing filter 52. The deodorizing filter 52 serves to suck odor particles. The deodorizing filter 52 is coated with a photocatalyst, such as titanium oxide (TiO₂). When ultraviolet rays are irradiated on the photocatalyst, the photocatalyst, which has a high oxidizing power, oxidizes a contaminant that is adhered to the objects to be deodorized. As a result, the contaminant is decomposed. This is generally referred to as a “photocatalytic action”. The oxidation of the contaminant by the photocatalyst lengthens the lifespan of the deodorizing filter 52. The ultraviolet rays, which have a strong bactericidal action, prevent contamination that would be generated due to the propagation of microorganisms, which may be dispersed from the deodorizing filter 52 so as to maintain a cleanliness of the deodorizing filter 52.

The filter case 51 includes a main body 51a, into which the deodorizing filter 52 is inserted, and a front cover 51b that is detachably attached to the main body 51a. UV-LED lamps 53 to irradiate ultraviolet rays toward the deodorizing filter 52 are installed on the inner surface of the front cover 51b. The front cover 51b is made of a semitransparent plastic. This allows a user to view the oxidation of the contaminant by the ultraviolet rays irradiated from the UV-LED lamps 53 through the front cover 51b. An inlet 54, through which air is introduced from the blowing duct 32 into the filter case 51, is formed through the rear surface of the upper portion of the main body 51a. An outlet 55, through which air filtered by the deodorizing filter 52 is exhausted to the outside of the washing machine therethrough, is formed through the lower portion of the front cover 51b. A filter net to filter out comparatively large-sized particles of the contaminant is installed in each of the inlet 54 and the outlet 55.

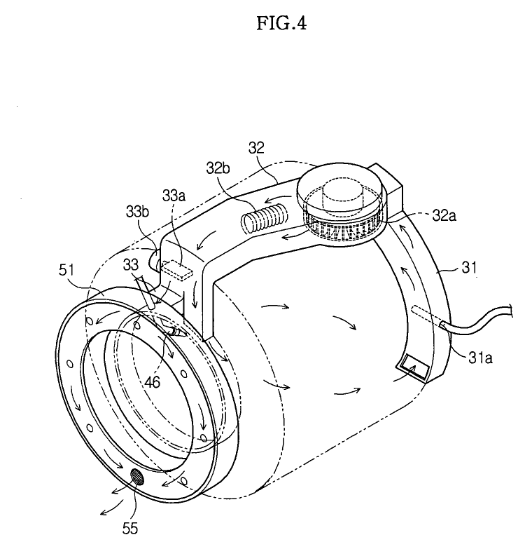

Now, with reference to FIG. 4, a structure of a branch duct 33 will be described. In order to circulate the air containing the odor particles into the deodorizing unit 50, the branch duct 33 in communication with the inlet 54 of the filter case 51 is branched from the front end of the blowing duct 32. A damper 33a, to control the branch duct 33 to be opened only in the deodorization mode, is installed in the branch duct 33, and a damper-driving motor 33b, to drive the damper 33a, is installed outside the branch duct 33.

Now, a deodorizing method of the washing machine having the deodorizing filter in accordance with the above embodiment of the present invention will be described.

Schematically, after particles generating offensive odor, such as the smell of food, tobacco, etc., are separated from objects permeated by the offensive odor, air containing the separated odor particles passes through the deodorizing unit 50, so that the deodorizing filter 52 may filter out odor particles.

Methods of separating the odor particles from the objects to be deodorized are as follows. A first method involves applying mechanical impacts to the objects to be deodorized. In the first method, when the rotary tub 22 containing the objects is rotated, the odor particles are separated from the objects by mechanical impact, such as frictional force between the objects or between the objects and the rotary tub 22 and impact generated by the falling of the objects in the rotary tub 22. A second method is that heat is applied to the objects to be deodorized. In the second method, when the heater 32b is operated and then the air blast fan 32b is operated, a hot draft heated by the heater 32b is supplied to the inside of the drum 20. The hot draft contacts the objects to be deodorized, and induces movement of the odor particles stuck to the objects so as to separate the odor particles from the objects. A third method is that odor particles that permeate the objects to be deodorized are dissolved in water. In the third method, when a small quantity of water in mist form is supplied to the inside of the drum 20 through the water feed nozzle 46, the odor particles stuck to the objects are dissolved in water and then are vaporized together with the water so as to be separated from the objects. One of the above three methods may be selectively used or two or more of the three methods may be combined so as to improve the deodorizing effect. Since the odor particles permeating the objects to be deodorized have different adhesive strengths to the objects based on the type of odor particle, in an embodiment of the invention, the above methods are suitably combined based on the adhesive strengths of the odor particles to the objects.

The odor particles separated from the objects by the above methods float in the drum 20. In order to prevent the separated odor particles from being re-adhered to the objects, the separated odor particles are discharged to the outside of the drum 20.

When the air blast fan 32a is operated, air containing the odor particles in the drum 20 is circulated via the duct 30. As shown by the arrows in FIG. 4, the air containing the odor particles, which is drawn into the condensing duct 31 in communication with the lower part of the fixed tub 21, is introduced into the blowing duct 32 so that the air passes through the air blast fan 32a and the heater 32b and enters into the upper portion of the front part of the inside of the fixed tub 21 communicating with the blowing duct 32. Here, a part of the air, having passed through the blowing duct 32, flows toward the branch duct 33, and when the damper-driving motor 33b is driven to open the damper 33a, the above part of the air is drawn into the deodorizing unit 50. The odor particles of the air drawn into the deodorizing unit 50 are filtered out by the deodorizing filter 52, and the purified air is exhausted to the outside of the washing machine through the outlet 55.

When the above part of the air circulated in the drum 20 is exhausted to the outside of the washing machine, the inside of the drum 20 has a pressure that is lower than that of the outside of the drum 20. Thus, the inside of the drum 20 is filled with new air through gaps formed along the circumference of the drum 20. The gaps allow the new air to compensate for the lowered pressure of the inside of the drum 20. The remainder of the air, not having passed through the branch duct 33, is again drawn into the drum 20 and recirculated in the drum 20. As described above, most of the odor particles, which are separated from the objects to be deodorized, are filtered out by the deodorizing filter 52 via a control of the circulation of the air containing the odor particles to allow the objects to be deodorized.

In case that the UV-LED lamps 53 are operated in the deodorization mode, the deodorizing filter 52 performs a photocatalytic action to oxidize the odor particles and other contaminants. Where the deodorizing filter 52 loses a deodorizing capacity thereof due to a use of the deodorizing filter 52 over a long period of time, the deodorizing filter 52 is either replaced with a new deodorizing filter or is washed and is then reused. Whether the deodorizing filter 52 is replaced or washed and reused is a function of the type of filter the deodorizing filter 52 is. Where the deodorizing filter 52 is an activated carbon filter, the deodorizing filter 52 should be replaced. Meanwhile, where the deodorizing filter 52 is a ceramic filter, the deodorizing filter should be simply washed and reused.

Although the separation of the odor particles from the objects to be deodorized and the removal of the separated odor particles using the deodorizing filter were sequentially described in the above noted embodiment of the present invention for convenience, the two operations are substantially performed simultaneously.

As is apparent from the above description, the present invention provides a washing machine having a deodorizing filter, which removes particles of offensive odor from laundry without operating wash, rinse and/or dehydration modes, and a deodorizing method thereof.

Thereby, the washing machine and the deodorizing method of the present invention reduce electricity and water consumption and prevent the deformation of the laundry generated by the washing.

Although a few embodiments of the present invention have been shown and described, it would be appreciated by those skilled in the art that changes may be made in these embodiments without departing from the principles and spirit of the invention, the scope of which is defined in the claims and their equivalents.

1. A washing machine comprising:
a deodorizing tub, into which objects to be deodorized are put;
a duct to communicate with the deodorizing tub and to serve as a passage through which air containing odor particles, which are separated from the objects in the deodorizing tub, passes; and
a deodorizing unit to remove the odor particles from the air passing through the duct.

2. A washing machine comprising:
a deodorizing tub, into which objects to be deodorized are put;
a duct to communicate with the deodorizing tub and to serve as a passage through which air containing odor particles, which are separated from the objects in the deodorizing tub, passes; and
a deodorizing unit, including a deodorizing filter to filter out and remove the odor particles from the air passing through the duct.

3. The washing machine according to claim 2, wherein the deodorizing unit further includes a photocatalyst to perform a photocatalystic action so as to oxidize the odor particles in the deodorizing filter, and UV-LED lamps.

4. The washing machine according to claim 2, wherein the duct, in communication with the deodorizing tub, circulates the air inside the deodorizing tub, and a branch duct is branched from the duct such that a part of the circulated air passes through the deodorizing unit, and communicates with the deodorizing unit.

5. The washing machine according to claim 4, wherein the branch duct is provided with a damper to open and close the branch duct.

6. The washing machine according to claim 4, wherein:
the deodorizing unit further includes a filter case to form a passage for the air flowing along the deodorizing filter so that the air containing the odor particles introduced from the branch duct is purified by the deodorizing filter; and
the filter case is provided with an outlet, through which the air purified by the deodorizing filter is exhausted to the outside of the washing machine.

7. The washing machine according to claim 6, wherein the deodorizing unit is installed on a door of the washing machine.

8. The washing machine according to claim 4, wherein:
the duct includes a condensing duct to communicate with a rear surface of the deodorizing tub, and a blowing duct, to communicate with the condensing duct, to be positioned on an upper surface of the deodorizing tub;
the blowing duct is provided with an air blast fan to generate power so as to circulate the air passing through the blowing duct, and a heater to heat the air passing through the blowing duct; and
the branch duct is branched from the blowing duct.

9. The washing machine according to claim 2, wherein the deodorizing filter is a ceramic filter or an activated carbon filter.

10. The washing machine according to claim 3, wherein the deodorizing filter is coated with the photocatalyst.

11. A deodorizing method of a washing machine comprising:
separating odor particles from objects to be deodorized; and
removing the separated odor particles by filtering out the separated odor particles with a deodorizing filter.

12. The deodorizing method according to claim 11, wherein the separating comprises at least one of supplying a hot draft to the objects to be deodorized, applying mechanical impact to the objects to be deodorized, or dissolving the odor particles in moisture that is supplied to the objects to be deodorized in a mist.

13. The deodorizing method according to claim 6, wherein in the removing, the air in the deodorizing tub, in which the separated odor particles float, is circulated into a duct communicating with the deodorizing tub, and a part of the circulated air containing the separated odor particles are purified by the deodorizing filter.

14. The deodorizing method according to claim 13, further comprising performing a photocatalystic action to oxidize the odor particles in the deodorizing filter.

15. The deodorizing method according to claim 14, further comprising exhausting the air that is purified by the deodorizing filter to the outside of the washing machine.

16. A washing machine, including a deodorizing tub having a fixed tub and a rotary tub into which objects to be deodorized are placed, comprising:
a duct to communicate with the deodorizing tub through which air containing odor particles, which are separated from the objects in the deodorizing tub, passes; and
a deodorizing unit to filter out and remove the odor particles from the air passing through the duct.

17. A washing machine, including a deodorizing tub having a fixed tub and a rotary tub into which objects to be deodorized are placed, comprising:
a duct to communicate with the deodorizing tub through which air containing odor particles, which are separated from the objects in the deodorizing tub, passes;
a branch duct, branched from the duct, through which a part of the circulated air that passes through the duct passes; and
a deodorizing unit to filter out and remove the odor particles from the air which passes through the branch duct.

18. A deodorizing method of a washing machine comprising:
separating odor particles from objects to be deodorized such that the odor particles are carried in an air flow; and
removing the odor particles by filtering the air flow with a deodorizing filter.

19. A washing machine, including a deodorizing tub having a fixed tub and a rotary tub into which objects to be deodorized are placed, comprising:
a duct to communicate with the deodorizing tub through which air containing odor particles, which are separated from the objects in the deodorizing tub, passes; and
a deodorizing unit to filter out and remove the odor particles from the air passing through the duct, wherein the odor particles are separated and removed substantially simultaneously.

20. A deodorizing method of a washing machine comprising:
separating odor particles from objects to be deodorized such that the odor particles are carried in an air flow; and
removing the odor particles by filtering the air flow with a deodorizing filter, wherein the separating and the removing are performed substantially simultaneously.
